(19) United States
(12) Patent Application Publication
(10) Pub. No.: US 2026/0073588 A1
(43) Pub. Date: Mar. 12, 2026

(54) METHODS FOR RAPID DOSE VOLUME HISTOGRAM CALCULATION FOR RADIOTHERAPY

(71) Applicant: RefleXion Medical, Inc., Hayward, CA (US)

(72) Inventors: Foma MIRONENKO, Hayward, CA (US); Yevgen VORONENKO, San Jose, CA (US); Lingxiong SHAO, Saratoga, CA (US)

(21) Appl. No.: 19/310,779

(22) Filed: Aug. 26, 2025

Related U.S. Application Data
(63) Continuation of application No. PCT/US2024/017358, filed on Feb. 26, 2024.
(60) Provisional application No. 63/487,404, filed on Feb. 28, 2023.

(51) Int. Cl. G06T 11/20; A61N 5/10; G16H 30/20

(52) U.S. Cl. CPC G06T 11/26; G16H 30/20; A61N 5/1031; G06T 2200/24; G06T 2210/41; G06T 2210/56

(57) ABSTRACT

Disclosed herein are statistical methods that use randomized sampling to generate a precise dose volume histogram (DVH) without the computational intensity of subdivision methods that rely on super-sampling of a 3D dose image. One variation of a method rapidly generates a DVH from a dose image by generating a randomly spaced point cloud of arbitrary size for each voxel of a volume of interest (VOI). These methods may be used to help expedite radiotherapy treatment planning and/or facilitate clinician review of whether a radiation dose distribution is acceptable. These methods may also be used to facilitate the calculation of bounded DVH curves. In some variations, adaptive radiotherapy methods may use these rapidly-generated DVHs to update or adjust radiation delivery on the day of treatment.

### CROSS-REFERENCE TO RELATED APPLICATIONS

This application is a continuation of International Application No. PCT/US2024/017358, filed Feb. 26, 2024, which claims priority to U.S. Provisional Patent Application 63/487,404 filed Feb. 28, 2023, the disclosure of which is hereby incorporated by reference in its entirety.

### BACKGROUND

Dose-volume histogram (DVH) curves may be used by clinicians to analyze and approve radiotherapy treatment plans. A DVH curve provides information about the amount of radiation dose delivered to a volume-of-interest (VOI) in a patient, indicating the proportion of a VOI that receives a certain dose level. A treatment plan dose distribution presented as a DVH curve may be used to evaluate whether a proposed treatment plan would provide the desired dose to a tumor. A DVH curve may also be used before a treatment session as part of a quality assurance (QA) session that validates the ability of the radiotherapy system to deliver the desired dose to a tumor, and after a treatment session to report on the dose delivered during the treatment session. DVH curves may be generated for radiation-sensitive regions, such as organs-at-risk (OARs), so that a clinician may confirm that the radiation-avoidance regions are not irradiated beyond a desired safety threshold.

However, the accuracy of a DVH curve may vary significantly depending on the size and shape of the VOI and the dose grid resolution. One method of generating a DVH curve for a VOI based on a dose image having a certain dose grid resolution uses an over-sampling technique, which subdivides the dose grid into sub-voxels to provide dose values with greater granularity. Such subdivision methods for calculating a DVH can be computationally-intensive, since the number of computations increases rapidly with the size of the VOI (which is a 3D structure). Moreover, small targets may require large oversampling factors (i.e., super-sampling, more subdivisions per voxels of the dose image), without which the DVH may be grossly inaccurate. In the scenario where a sharp dose gradient crosses the VOI, a large super-sampling factor may be needed to accurately represent the dose “edge” for an accurate DVH. Accordingly, improved methods for calculating precise DVH are desired.

### SUMMARY

Disclosed herein are methods of generating dose volume histogram (DVH) curves using statistic-based randomized sampling techniques. The randomized sampling methods described herein may be asymptotically optimal and may outperform widely-used DVH calculation methods that use oversampling (i.e., grid subdivision). One variation of the method comprises generating a randomly spaced point cloud of arbitrary size for each volume-of-interest (VOI), interpolating the dose volume to obtain dose values at each point in the point cloud, and using point-in-polygon testing to generate a DVH curve. Generating a DVH using a randomly spaced point cloud of arbitrary size for each voxel of the VOI may result in a DVH with better precision than using an oversampling or subdivision method with the same number of samples per voxel. Rapid and precise calculation of DVH curves may facilitate the generation, updating, and adapting of radiotherapy treatment plans.

One variation of a method for generating a DVH curve for a VOI that is defined by at least one VOI contour may include generating a randomly spaced point cloud of arbitrary size for each voxel of the VOI, generating a VOI point cloud comprising points within the VOI contour, obtaining dose values at each point in the VOI point cloud, and generating a DVH curve by calculating a proportion of points in the VOI point cloud at discrete dose levels. Generating the randomly spaced point cloud of arbitrary size for each voxel of the VOI may include selecting a point cloud size k and assigning the k points of the point cloud to random locations within the voxel. The point cloud size k may be an integer greater than or equal to 1. Assigning the k points of the point cloud to random locations may include generating random locations from a random seed. In some variations, each voxel may have a different random seed, and/or the random locations of the k points may be different for each voxel. Generating the VOI point cloud may further include using point-in-polygon testing to exclude points outside the VOI contour. Optionally, generating the VOI point cloud may further include identifying border voxels that are along a boundary of the VOI contour and applying point-in-polygon testing to the border voxels. In some variations, obtaining dose values at each point in the VOI point cloud may use a dose image. The dose image may have dose values at a regular grid having dose values at each grid node, and calculating the dose value for the points in the VOI point cloud may include generating an interpolated dose image by interpolating dose values for regions between the grid nodes, and assigning a dose value to each point in the VOI point cloud based on its location on the interpolated dose image. Interpolating dose values for regions between the grid nodes may use nearest neighbor interpolation. In some variations, the regular grid may correspond with a DICOM CT image grid, and/or may have the regular grid has a coordinate origin. The VOI voxels may be registered to the regular grid of the dose image and/or the VOI contour may be registered to the regular grid of the dose image.

Generating a DVH curve may include plotting the proportion of sampled points at the discrete dose levels. In some variations, the method may further include generating a graphical user interface that comprises a plot of the DVH curve. For example, the graphical user interface may have the DVH curve overlaid on a bounded DVH, where the bounded DVH has a minimum DVH curve and a maximum DVH curve. Optionally, the method may further include comparing the DVH curve with the bounded DVH and generating a notification that indicates whether the DVH is within a range defined by the minimum DVH curve and the maximum DVH curve.

Another method for generating a DVH for a VOI defined by a VOI contour may include dividing the VOI into voxels that have a desired grid size, selecting a point cloud size k for each of the voxels of the VOI, assigning, for each voxel, the k points of the point cloud to random locations within the voxel, generating a VOI point cloud that includes the points of the point clouds located within the VOI contour, calculating a dose value for the points in the VOI point cloud using a dose image comprising a grid of dose values, calculating a proportion of points in the VOI point cloud at discrete dose levels, and generating a DVH curve by plotting the proportion of sampled points at discrete dose levels. Generating the VOI point cloud may include removing points that are outside the VOI contour, for example, using point-in-polygon testing. Optionally, some methods may include identifying border voxels that are along a boundary of the VOI contour and applying point-in-polygon testing to the border voxels. Assigning the k points of the point cloud to random locations may include generating random locations from a random seed, optionally, each voxel may have a different random seed, and the random locations of the k points may be different for each voxel. The point cloud size k may be an integer greater than or equal to 1. In some variations, calculating the dose value for each point in the VOI point cloud may include generating an interpolated dose image by interpolating dose values for regions between the grid (e.g., between the grid nodes), and assigning a dose value to each point in the VOI point cloud based on its location on the interpolated dose image. Interpolating dose values for regions between the grid may use nearest neighbor interpolation and/or linear interpolation. The grid of dose values may correspond with a DICOM CT grid. The grid of dose values may have a coordinate origin and/or the VOI contour may be aligned to the grid of dose values.

Optionally, some variations of methods may include generating a graphical user interface that comprises a plot of the DVH curve. The graphical user interface may have the DVH curve overlaid on a bounded DVH, where the bounded DVH has a minimum DVH curve and a maximum DVH curve. The method may include comparing the DVH curve with the bounded DVH and generating a notification that indicates whether the DVH is within a range defined by the minimum DVH curve and the maximum DVH curve, e.g., generating a notification if the DVH is outside of the rang defined by the bounded DVH.

Another method of generating a DVH curve for a VOI defined by a VOI contour may include generating a VOI point cloud comprising a randomly spaced point cloud of arbitrary size with uniform density for the VOI, obtaining dose values at each point in the VOI point cloud, and generating a DVH curve by calculating a proportion of points in the VOI point cloud at discrete dose levels. The VOI point cloud comprises a plurality of points within the VOI contour. Obtaining dose values at each point in the VOI point cloud may use a grid of dose values.

### BRIEF DESCRIPTION OF THE DRAWINGS

FIGS. 1A-1C are schematic depictions of one variation of an over-sampling (or subdivision) method for generating a dose volume histogram (DVH).

FIG. 2A is a flow chart representation one variation of a method using a randomly spaced point cloud of arbitrary size for generating a DVH.

FIG. 2B is a flow chart representation of one variation of a method using a randomly spaced point cloud of arbitrary size for generating a DVH.

FIGS. 3A-3C are conceptual graphical representations of one variation of a method for generating a VOI point cloud based on randomized sampling for calculating a DVH.

FIGS. 3D-3F are conceptual graphical representations of one variation of a method for generating a VOI point cloud based on randomized sampling for calculating a DVH.

FIGS. 4A-4D are conceptual graphical representations of one variation of a method for calculating the dose values of the points in a VOI point cloud.

FIG. 5 is a plot from a simulation experiment of DVH generation, comparing DVH curves generated using a subdivision method with a DVH generated using a random sampling method.

FIG. 6A is a plot from a simulation experiment comparing the error of DVH curves generated using a subdivision method with the error of DVH curves generated using a random sampling method for VOIs of various shapes.

FIG. 6B is a table from a simulation experiment that depicts the volume percent error for DVH curves calculated using a subdivision method as compared to DVH curves calculated using the random sampling methods disclosed herein.

FIG. 7A is a depiction of one example of a dose image with incomplete dose information and an example VOI contour.

FIG. 7B is a flowchart representation of one variation of a method for assigning dose values for points within a VOI point cloud using a dose image with incomplete dose information.

FIGS. 7C-7D are depictions of one example of a dose image with incomplete dose information and an interpolated dose image generated using the method of FIG. 7B.

FIG. 7E depicts an optional graphical user interface depicting a second interpolated dose image.

FIGS. 7F-7G are DVH curves that were generated during a simulation comparing the results of calculating dose distributions using an incomplete dose image and the results of calculating dose distributions using a complete dose image.

FIGS. 8A-8H depict an example of calculating a family of DVH curves for a target region moving within a VOI.

FIGS. 9A-9H depict an example of calculating a family of DVH curves for a target region moving within a VOI that also has an organ-at-risk (OAR).

### DETAILED DESCRIPTION

Disclosed herein are statistical methods for fast DVH calculation based on randomized sampling. One example of a method for fast, precise DVH calculations using randomized sampling comprises generating a randomly spaced point cloud of arbitrary size for each voxel of the VOI, generating a VOI point cloud that includes points that are within the VOI contour, using a dose image to obtain dose values for each point of the VOI point cloud, and generating DVH by calculating the proportion of points in the VOI point cloud that are at discrete dose levels. In some variations, the size of the point cloud for each voxel of the VOI may be the same (i.e., the number of points that are sampled for each voxel is the same), however the placement or location of each point within a voxel is randomly generated. The random locations of the points in each voxel may differ from each other. After the random location of the points have been selected for each voxel, some variations may comprise determining which of those points are within the contours of the VOI by using a point-in-polygon testing technique. The points that are outside the VOI contour may be excluded and the remaining points may comprise the VOI point cloud. The dose value for each point in the VOI point cloud may be calculated based on a dose image. A dose image may comprise an array (or grid) of dose values, where the grid corresponds with the voxels of the VOI. In cases where the points of the VOI point cloud are located between the nodes of the dose grid (e.g., dose grid nodes, voxel centers), the dose value at those points may be determined by interpolating between the dose values on the grid of the dose image. The interpolation method may be linear interpolation and/or nearest neighbor interpolation. It should be understood that any appropriate interpolation method may be used to create an interpolated dose image which may then be used to determine the dose values at each point in the VOI point cloud.

The inputs to the methods described herein may comprise a VOI contour that defines the boundaries of the volume and a 3D dose information, such as a dose image. A 3D dose image may comprise an array or grid of dose values (which may be referred to as a dose grid), which provides dose information at regularly spaced intervals. In some variations, the dose grid may correspond with an image grid of a patient image. This may allow the dose values of the 3D dose image to be mapped onto the patient image. For example, the dose grid may correspond with a DICOM CT image grid that has x, y, z coordinates and a coordinate origin. The dose values on the dose grid may correspond to the dose values at the centers of the image grid. In some variations, the 3D dose image may be generated by a dose calculation module, and provided to a DVH generation module for use in calculating a DVH. It should be understood that the methods described herein are for a 3D volume, but for the sake of simplicity and clarity, the conceptual representations are in 2D. Although the figures depict 2D grids and pixels, the methods are used for processing 3D grids and voxels, and are therefore referred to as voxels throughout. In addition, the calculation of a single DVH for a single VOI is described herein, but it should be understood that these methods may be used to calculate DVH curves for multiple VOIs, including targets (e.g., tumors) and organs-at-risk (e.g., heart, lung, kidney, etc.).

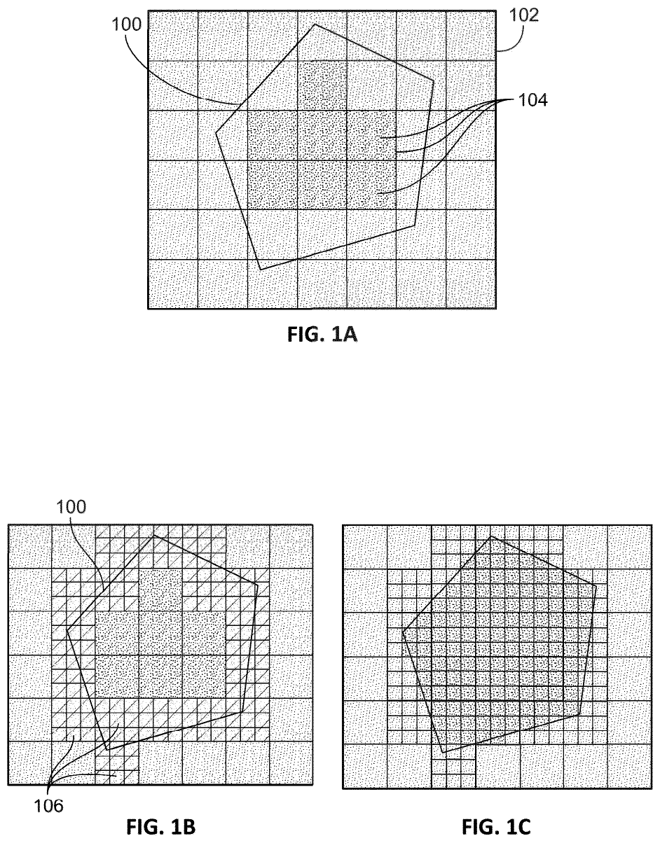

FIGS. 1A-1C schematically depict one implementation of an over-sampling method (which may also be referred to as a subdivision method). FIG. 1A depicts a VOI 100 on a grid 102 of voxels 104 (conceptually depicted as sub-pixels) that corresponds to the grid of a dose image (i.e., a dose grid). The over-sampling (or subdivision) method divides the voxels 104 into sub-voxels. FIG. 1B depicts one example where voxels on the VOI contour/boundary are divided into sub-voxels 106 while the internal voxels are not divided. FIG. 1C depicts another example of a subdivision method that also divides the internal voxels into sub-voxels. By increasing the number of sub voxels per voxel, the DVH calculation can be more accurate as compared to a lower number of sub voxels per voxel. However, subdivision methods for calculating an accurate DVH can be computationally-intensive, since the number of computations increases rapidly with the size of the VOI (which is a 3D structure). The randomized sampling methods described herein may be able to generate DVH curves similar levels of accuracy as those generated by subdivision methods but with far fewer samples per voxel and significantly reduced computational intensity.

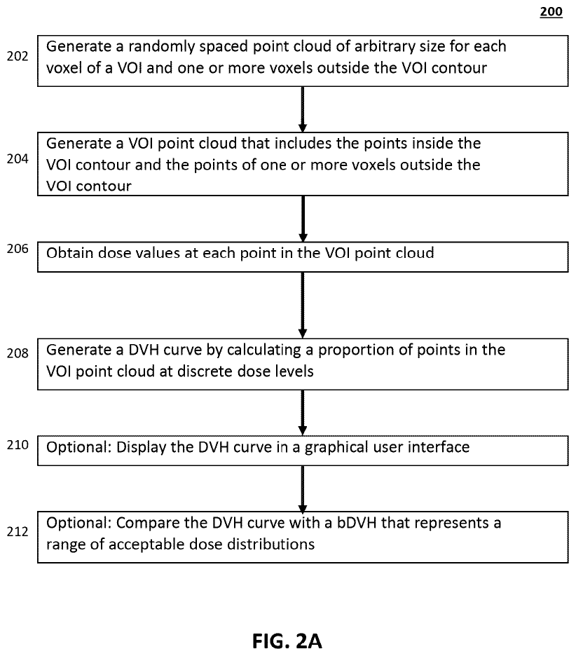

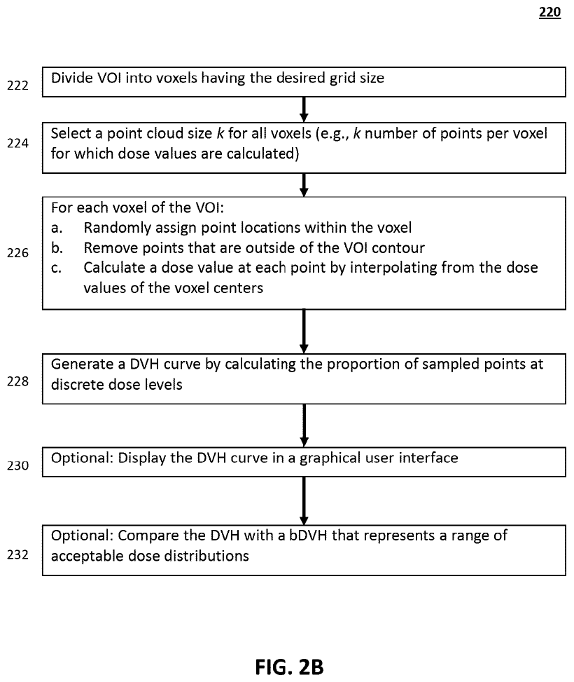

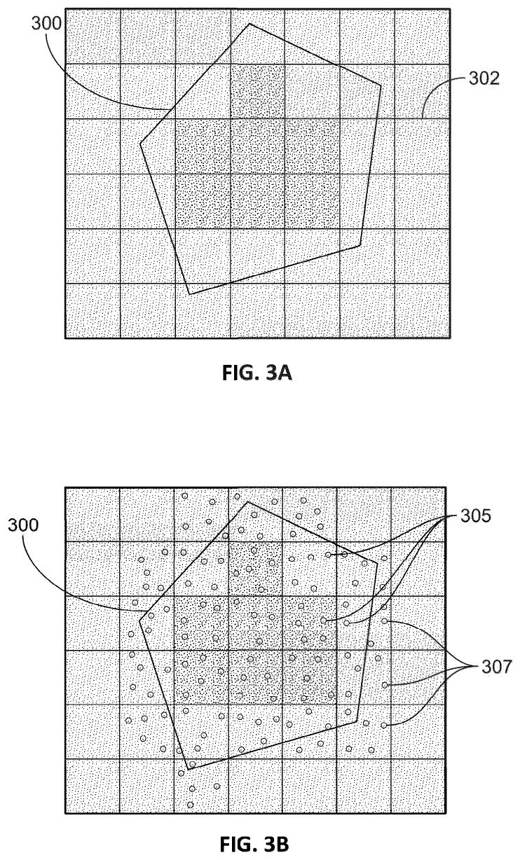

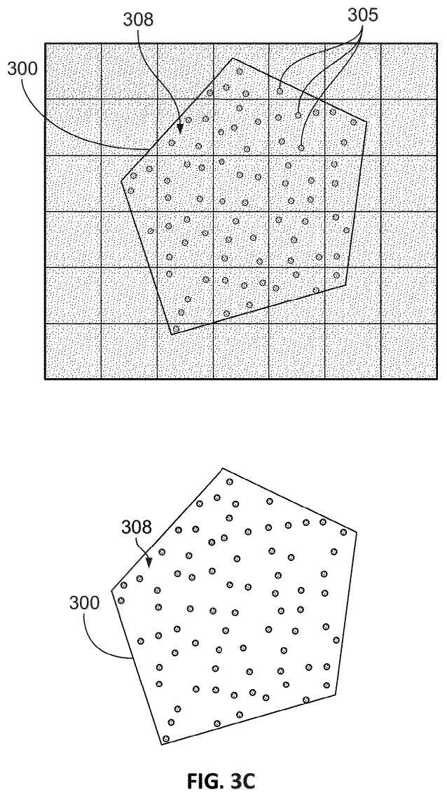

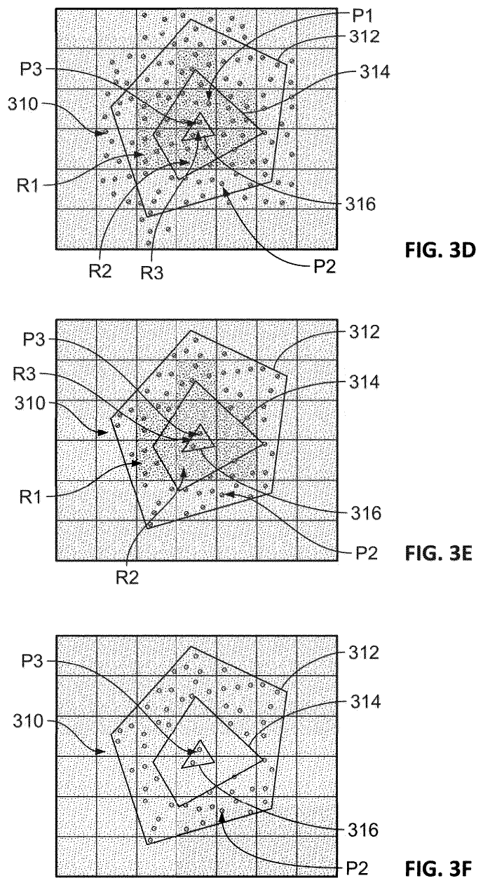

FIG. 2A depicts a flow chart of one variation of a randomized sampling method for generating a DVH. Method 200 comprises generating 202 a randomly spaced point cloud of arbitrary size for each voxel of a volume of interest (VOI) and/or generating 204 a VOI point cloud that includes the points inside the VOI contour and the points of one or more voxels outside the boundary of the VOI contour, obtaining dose values at each point in the VOI point cloud 206, and generating 208 a DVH curve by calculating a proportion of points in the VOI point cloud at discrete dose levels. Generating 204 a VOI point cloud may comprise expanding the VOI contour to include all the points in all of the voxels of a VOI (including the voxels on the boundary of the VOI contour) and one or more voxels outside the (initial) VOI contour. The VOI point cloud would then include all the points inside the expanded VOI contour. In some variations, the voxels included in an interpolation operation may be inside and outside the VOI contour, and/or include voxels of an expanded VOI contour. The number of voxels that a VOI contour may be expanded may be selected based upon the amount of voxel data needed for the interpolation operation. In some variations, obtaining dose values at each point in the VOI cloud uses a dose image. FIG. 3A is a conceptual depiction of a VOI contour 300 that is overlaid on a dose grid 302 (e.g., a grid of a dose image). FIG. 3B is a conceptual depiction of generating a randomly spaced point cloud for each voxel that corresponds with the VOI. As depicted in FIG. 3B, there are 5 points sampled for each voxel; in other words, the size of each point cloud is 5. While this example shows that there are 5 points sampled for each voxel it should be understood that any integer number greater than or equal to one may be selected, for example, 7, 8, 20, 27, 64, 125, etc. Generating 202 a randomly spaced point cloud of arbitrary size may include selecting the size of the point cloud; that is the number of points to be sampled within each VOI voxel, and then assigning the points to random locations within the VOI, as shown in FIG. 3B. In some variations, the point cloud may include points inside the voxels of a VOI contour that has been expanded by one or more voxels. In some variations, assigning the points of the point cloud to random locations within a voxel includes generating the random locations from a random seed. The locations of the points in each voxel may differ from the locations of the points in the other voxels. In some variations, the random point locations for each voxel may be generated independently from each other. For example, random point locations may be generated using a random number generator provided with a random seed, and the random seed for each voxel may be different to generate different point locations for each voxel.

As can be seen in FIG. 3B, some of the points in the point cloud of a voxel are inside the contour (i.e., boundary) of the VOI (e.g., points 305) while some of the points are outside the contours of the VOI (e.g., 307). Method 200 may comprise determining 204 which of the points are inside the contour of the VOI and excluding the points that are outside the contour of the VOI. In some variations, point-in-polygon testing may be used to determine which points are inside or outside the contour of the VOI. The points that are located outside the contour of the VOI are excluded from the VOI point cloud. In some variations, the VOI point cloud only includes the points that are within the contour of the VOI. FIG. 3C provides conceptual depictions of the VOI point cloud 308 after the points that are outside of the VOI contour have been removed (top panel includes the VOI voxels/grid while the bottom panel does not have the VOI voxels/grid). The dose values of the points in the VOI point cloud 308 are the sampling points that will be used to calculate the DVH. In some variations, the voxels included in an interpolation operation may expand beyond the VOI contour. For example, to the extent that interpolation is used to determine the dose value at each point in the VOI point cloud, those dose values may be interpolated from dose values of voxels just outside and/or adjacent to the VOI contour (e.g., one or more immediate neighboring voxels outside the VOI contour), i.e., interpolated from voxels of an expanded VOI contour. Different types of interpolation may require different amounts of VOI contour expansion. The number of voxels that a VOI contour may be expanded may be selected based upon the amount of voxel data needed for the interpolation operation.

In the example of FIGS. 3A-3C, VOI 300 has a single VOI contour that encloses a continuous region for which a DVH is to be calculated. However, in some other examples, a VOI may have multiple contours, including “nested” contours. This may arise in situations where a tumor or an OAR has a ring-shape, such as a tumor that encircles the spine. More generally, some VOIs, such as lung tumors, may have irregular geometries where certain regions within the VOI may require a certain dose amount while other regions require a different dose amount. FIGS. 3D-3F depict an example of a VOI 310 that has three contours 312, 314, 316 that define three regions R1, R2, R3. Contour 314 is within contour 312, and contour 316 is within contour 314. Region R3 is a region that is enclosed by contour 316, i.e., the interior of contour 316. Region R2 is a region that is enclosed by contour 314, but excludes the interior of contour 316. Region R1 is a region that is enclosed by contour 312, but excludes the interior of contour 314. In this example, the VOI comprises regions R1 and R3, but not region R2. Method 200 may be used to generate a DVH for VOI 310 as well, with additional processing to determine which points in the voxels are in the VOI (i.e., regions R1 and R3), so that the VOI point cloud does not include any points that are in region R2. As depicted in FIGS. 3D and 3E, the points in region R2 have been determined not to be part of VOI 310 (i.e., not in regions R1 and R3) and have been excluded. One variation of determining whether a point is within VOI 310 is testing each point of the point cloud (e.g., the points within the VOI contour 312) and determining whether it is within an odd number of contours or an even number of contours. If a point is in an even number of contours (e.g., point P1 is within contours 312 and 314—for a total of 2 contours), then that point is not part of the VOI 310. However, if a point is in an odd number of contours (e.g., point P2 is within one contour-contour 312, point P3 is within 3 contours—contours 312, 314, 316), then that point is part of the VOI 310. FIG. 3F depicts the resultant VOI point cloud, which excludes points that are not in VOI 310 (i.e., excludes the points in R2).

Alternatively, instead of generating VOI point cloud on a voxel-by-voxel basis and then eliminating any points that are not within the VOI contour, method 200 may comprise generating a VOI point cloud by generating a randomly spaced point cloud of arbitrary size with uniform density within the VOI contour. Although the average density of points in the VOI point cloud is uniform across the VOI, the location of the individual points may be randomly spaced throughout the VOI (e.g., using a random number generator with a selected random seed). In some variations, generating a VOI point cloud within a VOI contour may comprise generating a point cloud of arbitrary size where the points are randomly spaced, defining a bounding box that has a shape that corresponds to (e.g., approximates) the shape of the VOI contour, and generating a point cloud that comprises only the points that are within the defined bounding box. That is, the point cloud depicted in the bottom panel of FIG. 3C may be generated without dividing VOI 300 into pixels or voxels. The points of the VOI point cloud may be uniformly distributed, while in other variations, the points may not be uniformly distributed. For example, a VOI point cloud may have regions of increased point density (e.g., toward the boundary of the VOI contour) or decreased/reduced point density (e.g., in regions of relatively uniform radiation dose). For point clouds with non-uniform point density, this non-uniformity may be accounted for while calculating the proportion of points at discrete dose levels (e.g., step 208 of FIG. 2A) by weighting the dose values of those points using a weight factor that is inversely proportional to its density. The bounding box may be defined as the contour of VOI 300, and any point generation method may be used to generate a point cloud within VOI 300. One variation of a point generation method may comprise generating a non-uniformly distributed set of points, where the point density increases toward the boundary of the bounding box contour, and/or in regions of the bounding box with a higher dose gradient. Alternatively, or additionally, a variation of a point generation method may comprise generating a uniformly distributed set of points and subdividing the image voxels into finer grids in regions at or near the boundary of the bounding box contour and/or in region of the bounding box with a higher dose gradient. Subdividing the image voxels into finer grids (i.e., to have a small voxel size) result in more points per voxel region, which may result in a higher number or density of points at or near the bounding box boundary and/or regions with a higher dose gradient. In some variations, the number of points (e.g., from about 100 points to about 10,000 points or more) and the density of points (whether uniform density or non-uniform density) may be defined for a point generation method, which then generate a random point cloud within the boundary of the bounding box (e.g., the VOI contour).

In some variations, a dose image may only have dose values at regularly spaced intervals on the dose grid, and may not have dose values for the regions between the grid. In this example, the dose image may only have dose values at the center of each voxel, and may not have dose values for the points in the VOI point cloud that are not at the center of each voxel. To obtain the dose values for the points in the VOI point cloud, one variation may comprise interpolating the dose values for regions between the dose grid nodes (e.g., voxel centers). This interpolated dose image may then be used to determine the dose values for the points in the VOI point cloud based on their location on the interpolated dose image.

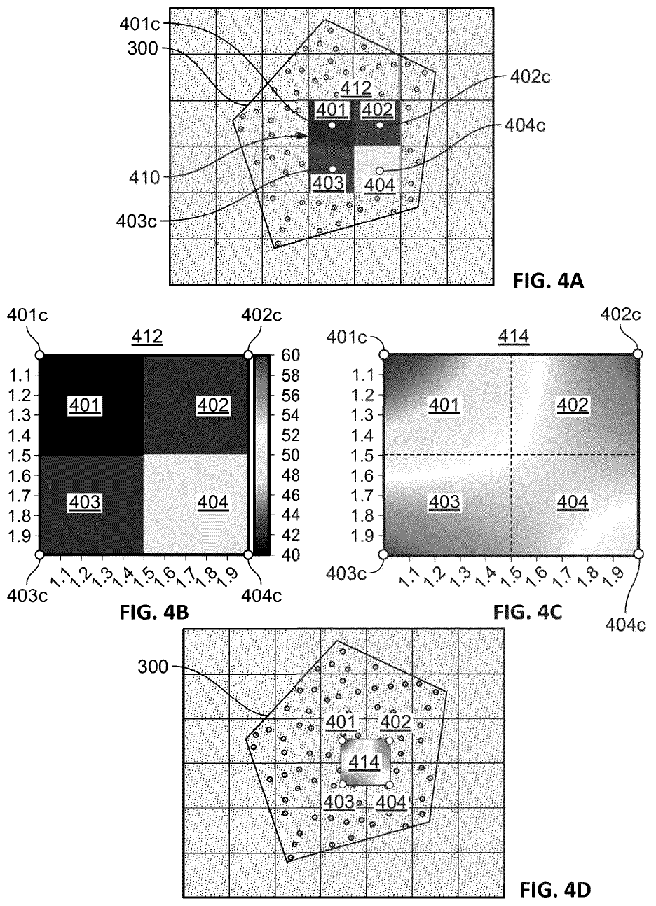

FIGS. 4A-4D depict graphical conceptual diagrams of one variation of a method for using a dose image, which may only have dose values at regularly spaced intervals on a grid, to generate an interpolated dose image, which may have dose values for regions between the grid. This method may be used in method 200 to obtain 206 dose values at the points in the VOI point cloud. FIG. 4A depicts a VOI 300 (similar to the VOI depicted in FIGS. 3A-3C above) with a VOI point cloud. As an illustration, the interpolation of the regions between a cluster 410 of four voxels (401, 402, 403, 404), is depicted in FIGS. 4A-4D. Interpolation of other regions in the VOI may be performed in a similar fashion. In this example, the dose image contains dose values for the center location of the voxels 401c, 402c, 403c, 404c; that is, the dose grid has nodes that correspond to the center of the voxels and only has dose values at the nodes. For clarity, only the portion of the dose image 412 that corresponds to the voxel cluster 410 is depicted. Also for the sake of clarity, the points of the VOI point cloud within the voxel cluster 410 are not shown. Each of the voxels in the cluster 410 have a voxel center 401c, 402c, 403c, 404c. FIG. 4B depicts the voxel centers 401c, 402c, 403c, 404c, and the regions of the voxels that form a convex hull between the voxel centers. Each of the voxel centers 401c, 402c, 403c, 404c has a dose value as specified by the dose image, which dose value is represented by the colors of each voxel in FIG. 4B. Any suitable interpolation method may be used to calculate the dose values for the voxel regions between the voxel centers 401c, 402c, 403c, 404c, for example, linear interpolation or nearest-neighbor interpolation. FIG. 4C depicts an example of an interpolated dose image 414 generated by interpolating the dose values at voxel centers 401c, 402c, 403c, 404c. As depicted in FIG. 4D, the interpolated dose image 414 may be combined with the VOI voxels and the dose values of the points of the VOI point cloud may be assigned based on the interpolated dose image 414. The interpolation method used to generate the interpolated dose image 414 for the voxel cluster 410 may be repeated for the remainder of the voxels of the VOI. The interpolated dose image for the entire VOI may then be used to obtain dose values at each point of the VOI point cloud.

Alternatively, or additionally, the dose at each point in the VOI point cloud may be assigned a dose value using a deterministic function that may analytically calculate a dose value based on known dose values and/or simulation models of a patient and/or a dose distribution. For example, some methods may comprise calculating a dose value at each point in the VOI point cloud by interpolating between known dose values. In some variations, a deterministic function may comprise a synthetic dose distribution (e.g., a non-zero dose value within the VOI contour and a zero dose value outside the VOI contour), such as a user-defined or “idealized” dose distribution that may be arbitrarily defined as desired.

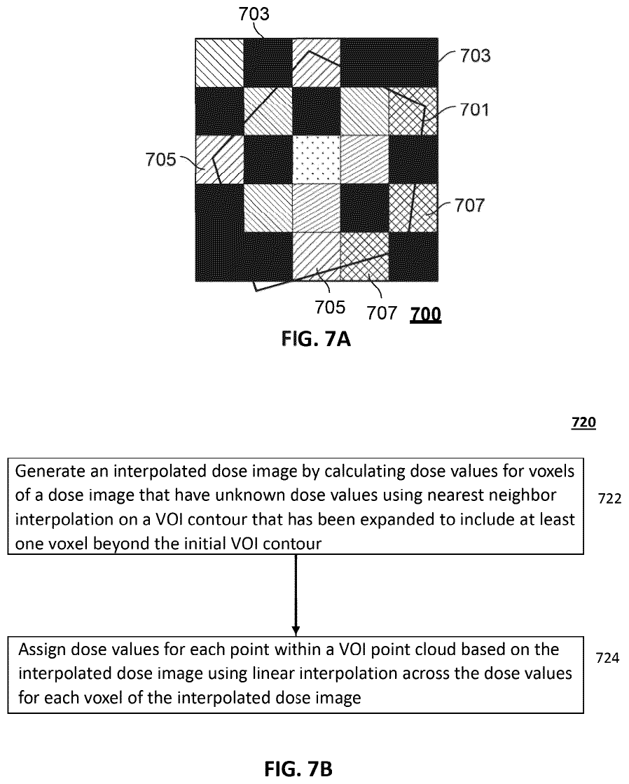

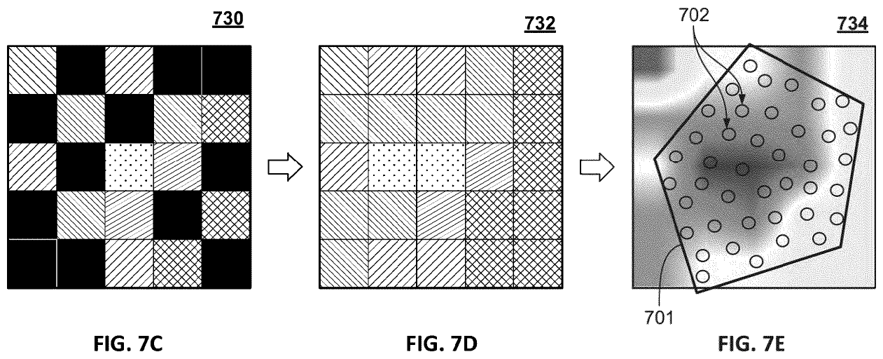

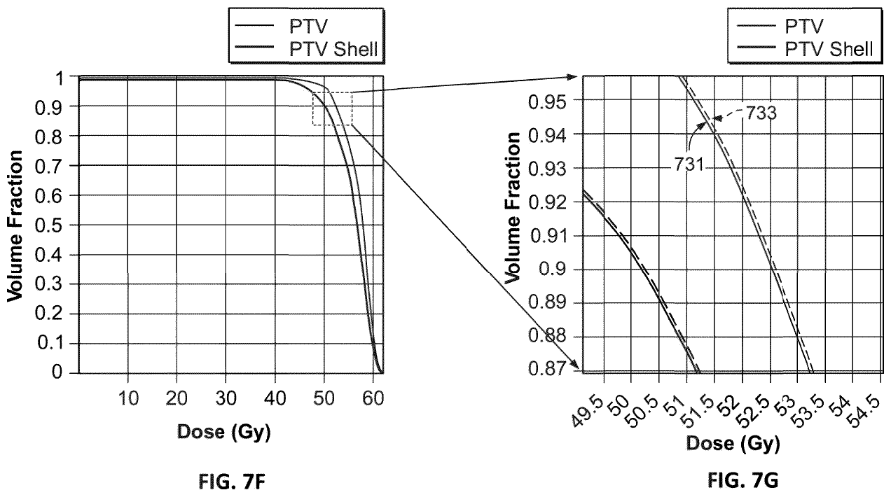

In some variations, such as during a fluence map optimization portion of treatment planning, a dose image may only have dose values for some voxels but not others. FIG. 7A depicts an example of a dose image 700 and a VOI contour 701 where dose values are unknown for certain voxels (e.g., black voxels 703) while dose values are known for other voxels (e.g., hashed voxels 705 and cross-hatched voxels 707). Since some voxels have unknown dose values, points of a VOI point cloud within those voxels may also have unknown dose values. One variation of a method to calculate dose values for the voxels with unknown dose values may comprise using nearest neighbor (NN) interpolation to calculate a dose value for voxels with unknown dose values, and then use linear interpolation to calculate the dose values for points within those voxels. Other interpolation methods of similar computational complexity to NN interpolation may be used, for example, k nearest neighbor (KNN) interpolation, weighted KNN interpolation, and/or a pre-trained neural network. When using any of these interpolation methods, the voxels included in the interpolation may expand beyond the (initial) VOI contour. For example, a linear interpolation operation may include voxels of a VOI contour that has been expanded by at least 1 voxel outside its boundary. A cubic interpolation may include voxels of a VOI contour that has been expanded by at least 2 voxels outside its boundary. The number of voxels that a VOI contour may be expanded by may be selected based upon the amount of voxel data needed for the interpolation operation. While other methods may use scattered point interpolation to calculate dose values for the points in the VOI point cloud, scattered point interpolation is a computationally intensive method. In contrast, a nearest neighbor interpolation (and variants thereof) and a linear interpolation are computationally efficient (e.g., nearest neighbor interpolation is about 10 times faster than scatter interpolation) and yet, provide results that are comparably accurate to scattered point interpolation. It is unexpected and surprising that the dual-interpolation method described herein which comprises two less computationally intensive interpolation methods could yield a result that is similarly accurate to a complex, computationally intensive interpolation method. The second interpolation of the dual-interpolation method may be linear interpolation. However, it should be understood that it may be any standard interpolation method, such as cubic interpolation, spline interpolation, polynomial interpolation, and/or Fourier interpolation.

FIG. 7B is a flowchart representation of one variation of method 720 for assigning dose values for points within a VOI point cloud using a dose image with incomplete or missing dose information. FIGS. 7C-7D conceptually depict an example of a dose image having voxels with missing or unknown dose values that is used to calculate dose values for a VOI point cloud using the method of FIG. 7B. Method 720 may comprise generating 722 an interpolated dose image by calculating dose values for voxels of a dose image that have unknown dose values using nearest neighbor interpolation on a VOI contour that has been expanded to include at least one voxel beyond the initial VOI contour, and assigning 724 dose values for each point within a VOI point cloud based on the interpolated dose image using linear interpolation across the dose values for each voxel of the interpolated dose image. The initial dose image may have some voxels with previously-known or calculated dose values and some voxels that do not have dose information. The voxels included in the linear interpolation operation may expand beyond the VOI contour. For example, the dose values for voxels with missing dose information may be interpolated from dose values of voxels outside and/or adjacent to the VOI contour (e.g., one or more immediate neighboring voxels outside the VOI contour), i.e., interpolated from voxels of an expanded VOI contour. Linear interpolation may utilize dose values from voxels that are within a VOI contour that has been expanded by at least 1 voxel. Different types of interpolation may require different amounts of VOI contour expansion. The number of voxels that a VOI contour may be expanded by may be selected based upon the amount of voxel data needed for the interpolation operation. FIG. 7C depicts an example of such a dose image 730, where the voxels with unknown dose values are shaded black. The dose values for the voxels without dose information may be determined by applying nearest neighbor interpolation to the voxels with known dose values. The result is an interpolated dose image 732 where all of the voxels have a known dose value, whether previously known or calculated using nearest neighbor interpolation, as depicted in FIG. 7D. Now, with the interpolated dose image 732 where all voxels have a known dose value, individual points in the VOI point cloud 702 may be assigned a dose value by linear interpolation of the known dose values of voxels of the interpolated dose image 732.

The VOI contour 701 may be superimposed on the interpolated dose image 732 and displayed to the user in a graphical user interface. Optionally, in some variations, a second interpolated dose image 734 may be generated by calculating (e.g., by interpolation) the dose values across the entire region of a voxel. FIG. 7E depicts an example of a second interpolated dose image 734 that may be generated from the first interpolated image 732 using any of the interpolation methods described herein.

FIGS. 7F and 7G are DVH curves that were generated during a simulation, where one DVH curve is generated using a dose plot with incomplete dose information and then processed according to the dual-interpolation method 720 depicted in FIG. 7B and another DVH curve is generated using a dose plot with complete dose information. The dose plot with incomplete dose information may be a dose plot that is generated during a treatment planning optimization (e.g., fluence map optimization) process, while the dose plot with complete dose information may be generated after the optimization process. DVH curves were generated for a target region (PTV) and a shell around the target region (PTV shell). The simulation generated a DVH curve (solid line) using the dual-interpolation method to calculate any missing dose information, and generated a DVH curve (dotted line) using a complete set of dose values, for each of the target region (thick lines) and the shell region (thin lines). FIG. 7F depicts the DVH curves for the simulation and FIG. 7G depicts a close-up view of the portion of the curve enclosed in the square dotted lines. As shown in FIG. 7G, the DVH curve (solid line 731) that was generated using the dual-interpolation method applied to a dose image with incomplete dose information very closely approximates the DVH curve (dotted line 733) that was generated using a dose image with complete dose information. The simulation demonstrated that for both the target region DVH and the shell region DVH, the dual-interpolation method applied to a dose image having incomplete dose information performs nearly as well as generating a DVH with a dose image having complete dose information. The ability to generate accurate DVH curves with good accuracy and low computational cost may facilitate the generation of DVH curves more frequently during treatment planning optimization. Generating DVH curves during the iterative process of optimization and displaying these curves to the user (e.g., a clinician and/or medical physicist or dosimetrist) may help improve the quality of radiotherapy treatment plans and/or reduce the amount of time to generate a treatment plan.

Once dose values at each point in the VOI point cloud have been defined using any of the methods described herein, method 200 comprises generating 208 a DVH curve by calculating a proportion of points in the VOI point cloud at discrete dose levels. In some variations, this may comprise counting the number of points at each discrete (or desired) dose level, calculating the proportion of points at that dose level over the total number of points (e.g., the volume fraction, percent volume), and plotting the proportion value for each dose level. Optionally, in some variations where the density of the VOI point cloud was not uniform, the non-uniform density may be accounted for by weighting the dose values of those points using a weight factor that is inversely proportional to its density.

After the DVH curve is generated, method 200 may optionally comprise displaying 210 the DVH curve in a graphical user interface, and may also optionally comprise comparing 212 the DVH curve with a bounded DVH that represents a range of acceptable dose distributions. A bounded DVH (bDVH) may have a minimum DVH curve and a maximum DVH curve. In some variations, the minimum DVH curve and the maximum DVH curve may define a dose distribution range that a clinician considers acceptable for that VOI. A DVH calculated using dose image generated on the day of treatment (e.g., based on image data acquired on the day of treatment) may be compared to the bDVH previously accepted by a clinician, and radiation delivery may only proceed if the DVH is within the bounds of the bDVH. In some variations, a DVH calculated using a dose image generated from updated imaging data may be used to evaluate whether a treatment delivery should be updated or adapted. Methods for calculating a bDVH are described further below.

FIG. 2B depicts a flowchart representation of one variation of a randomized sampling method for generating a DVH. Method 220 may comprise dividing 222 the VOI into voxels having the desired grid size, selecting 224 a point cloud size k for all voxels, and, for each voxel, randomly assigning 226a point locations within the voxel, removing 226b points that are outside of the VOI contour, and calculating 226c a dose value at each point by interpolating from the dose values of the voxel centers. In some variations, the voxels included in the interpolation may include voxels that are outside (e.g., beyond) the VOI contour. For example, interpolation when calculating 226c the dose value at each point may include voxels of an expanded VOI contour, where the “initial” VOI contour has been expanded by at least 1 voxel outside its boundary (e.g., 1 immediate neighbor voxel beyond the VOI contour). Other types of interpolation may require different amounts of VOI contour expansion to include sufficient voxel data for the interpolation. For example, a cubic interpolation may include voxels of a VOI contour that has been expanded by at least 2 voxels (e.g., 2 immediate neighbors) outside its boundary. The number of voxels that a VOI contour may be expanded may be selected based upon the amount of voxel data needed for the interpolation operation. Method 220 may comprise generating 228 a DVH curve by calculating the proportion of sampled points at discrete dose levels. Dividing 222 the VOI into voxels may include overlaying the VOI contour/boundary with a dose grid of the dose image, such as is depicted in FIG. 3A. In some variations, the dose grid may correspond with the grid of a patient image, such as a DICOM CT image or PET image. The point cloud size k may be the number of points per voxel for which dose values are calculated, and may be any integer value that is greater than or equal to 1, e.g., k=1, 7, 8, 20, 27, 64, 125. As described previously, randomly assigning 226a point locations within a voxel may use a random seed, which may differ across the voxels so that the random point locations in each voxel are different (see FIG. 3B). In some variations, point-in-polygon testing may be used to remove 226b points that are outside of the VOI contour (see FIG. 3C). Calculating the dose value at each point in the VOI contour may use a dose image and an interpolating method similar to the method described and depicted in FIGS. 4A-4D.

After the DVH curve is generated, method 220 may optionally comprise displaying 230 the DVH curve in a graphical user interface, and may also optionally comprise comparing 232 the DVH curve with a bounded DVH that represents a range of acceptable dose distributions. A bounded DVH (bDVH) may have a minimum DVH curve and a maximum DVH curve. In some variations, the minimum DVH curve and the maximum DVH curve may define a dose distribution range that a clinician considers acceptable for that VOI. A DVH calculated using dose image generated on the day of treatment (e.g., based on image data acquired on the day of treatment) may be compared to the bDVH previously accepted by a clinician, and radiation delivery may only proceed if the DVH is within the bounds of the bDVH. In some variations, a DVH calculated using a dose image generated from updated imaging data may be used to evaluate whether a treatment delivery should be updated or adapted.

The statistical methods for fast DVH generation based on randomized sampling, alone or in combination with the dual-interpolation dose image generation methods described herein may be used to generate DVH curves at various points in a radiotherapy workflow. For example, the randomized sampling methods described herein may be used to calculate a DVH during treatment planning, for example, during and/or after fluence map optimization, and/or after final dose calculation. If used during fluence map optimization, cost functions and/or constraints based on DVH curves may be used as part of optimization. These methods may be used to generate DVH curves as part of generating the minimum DVH and maximum DVH curves for a bounded DVH. In variations where the bDVH captures dose variations as a tumor (VOI and/or OAR) shifts position by generating a family of DVH curves that represent the dose to the VOI at different locations, the interpolated dose image (which was calculated for the first location of the tumor) and the randomly spaced point cloud for each voxel of the tumor may be re-used for each shifted tumor location. This way, the family of DVH curves for various tumor locations may be generated by “re-using” the point cloud calculation for each tumor voxel and greatly reduce the computation time for generating a bounded DVH.

The randomized sampling methods described herein may be used on the day of treatment to evaluate the safety and/or suitability of the radiotherapy treatment for the patient. For example, in biology-guided radiotherapy (BgRT), a PET scan is obtained with the patient on the radiotherapy system before the treatment begins. The PET scan is used to evaluate the quality of the PET signal to confirm that it is sufficient for BgRT treatment, and may also be used in conjunction with the localization CT (also taken on the same day with the patient on the BgRT radiotherapy system) to calculate a predicted dose image. The randomized sampling methods may be able to rapidly and precisely calculate a DVH based on this dose image, and display the DVH for comparison with a bound DVH that has been pre-approved by a clinician. This may shorten the amount of time a patient is on the radiotherapy platform, which may help a clinic to treat more patients. In addition, rapid and precise DVH curves may help determine whether a radiotherapy plan should be adapted and/or whether the radiation delivery should be modified.

### Simulation Experiments

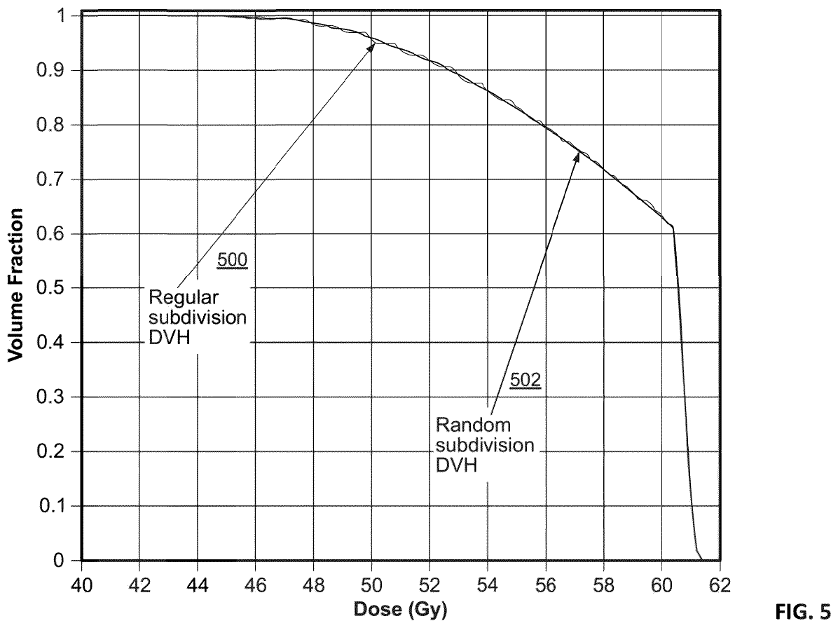

Simulation experiments and tests were conducted to compare the accuracy of calculating a DVH using a subdivision method (i.e., the method described and depicted in FIGS. 1A-1C) and the randomized sampling method disclosed herein. The simulation experiments provided the same VOI contour and dose image for the subdivision method and the randomized sampling method. FIG. 5 depicts a simulation experiment where a first DVH 500 was generated using a subdivision method and a second DVH 502 was generated using a randomized sampling method. In this experiment, the subdivision method used 125 samples per voxel (i.e., 125 sub voxels per voxel) and the randomized sampling method used 20 samples per voxel. Although the randomized sampling method used much fewer samples per voxel to generate the DVH as compared to subdivision method (20 samples vs 125 sub voxels), the accuracy of the resultant DVH curves appear to be quite similar. However, the DVH 502 generated using randomized sampling is smoother than the DVH 500 generated using the subdivision method. The irregular undulations and fluctuations of DVH 500 may cumulatively lead to large deviations (e.g., errors) of dose coverage of a VOI.

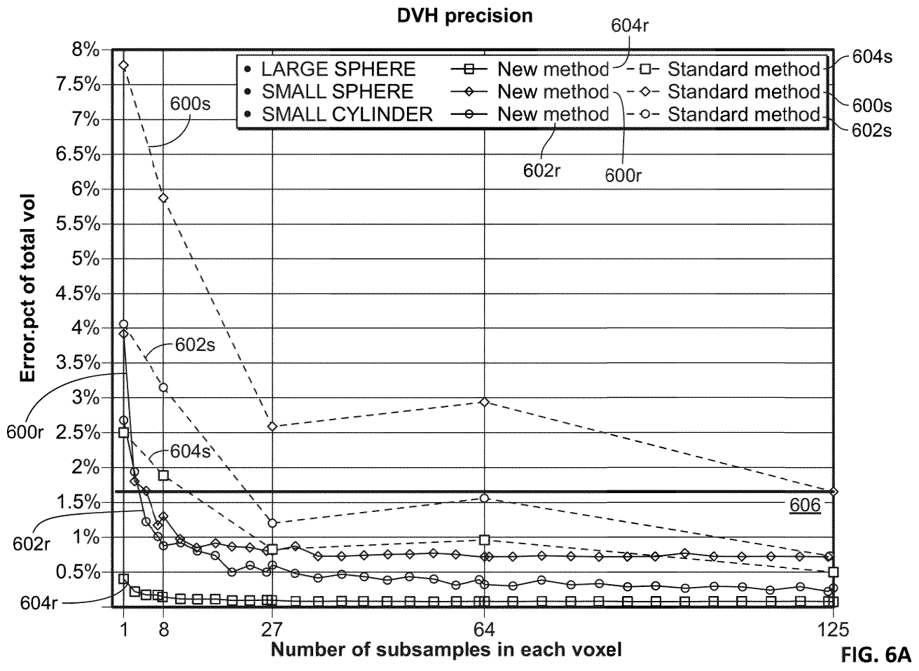

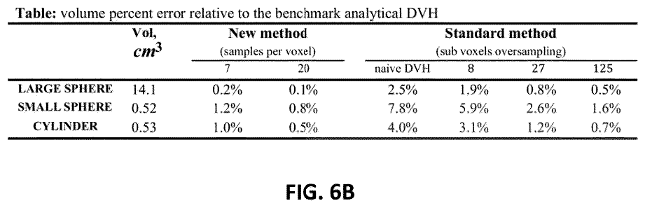

In another simulation experiment, the same VOI contour and dose image were provided for the subdivision method and the randomized sampling method, and the accuracy of the generated DVHs were compared with an analytical exact DVH (i.e., calculated based on an exact formula). FIGS. 6A and 6B depict the results of a simulation experiment comparing the DVHs calculated for different VOI having different shapes using a subdivision method and the randomized sampling methods described herein. In this simulation experiment, the subdivision method and the randomized sampling method were used to generate DVH curves for a large sphere (14137.2 mm³ volume, 1.5 cm radius), a small sphere (523.6 mm³ volume, 0.5 cm radius), and a small cylinder (538.8 mm³, volume, 0.3 cm xy radius). FIG. 6A depicts a graph of the error for DVH curves calculated using the randomized sampling methods described herein and a subdivision method for each of the large sphere, small sphere, and small cylinder. A dose distribution (i.e., dose image) on a 1 mm grid with a gradient along DICOM Z axis was used by both methods. The DVH curves generated by these two methods were compared to an analytical exact DVH curve to calculate the error percent of total volume (Y-axis of the plot on FIG. 6A). The number of samples per voxel comprise the X-axis of the plot. DVH accuracy for a chosen oversampling rate is defined as maximal absolute deviation of calculated volumes from the analytical DVH for the entire dose range, i.e., L_{∞} norm of the volume differences. Lines 600s and 600r represent the DVH error for a small sphere as calculated by the subdivision method and the randomized sampling methods, respectively. Lines 602s and 602r represent the DVH error for a small cylinder as calculated by the subdivision method and the randomized sampling methods, respectively. Lines 604s and 604r represent the DVH error for a large sphere as calculated by the subdivision method and the randomized sampling methods, respectively. Notably, comparing the DVHs for the small sphere along the line 606, the randomized sampling method resulted in a DVH curve with approximately a 1.6% error with 5 samples while the subdivision method needed 125 samples to attain the same error. Taking fewer samples per voxel to calculate a DVH curve while still maintaining comparable accuracy may greatly reduce the DVH calculation time, for example, from 1.25 sec to 0.1 sec for a single VOI. For 40 VOIs, the computation time may be reduced from 50 secs to 4 secs. Surprisingly, across all three VOIs, 8 sample points per voxel were enough for the randomized sampling method to output more accurate DVH curves than the subdivision method with 5³=125 sub voxels oversampling. This means that keeping computational error low, CPU time may be reduced by 15 times when generating DVH curves using the randomized sampling method as compared to subdivision method.

Turning to the table in FIG. 6B depicting results from the same simulation experiment as FIG. 6A, for the large sphere, the randomized sampling method (“New Method”) with 7 samples per voxel is much more accurate than the subdivision method (“Standard Method”), even in scenarios where the subdivision method uses many more samples per voxel (i.e., divides a voxel into many sub voxels). For the large sphere, the error for the subdivision method using 3³=27 sub voxels is 0.8% and 5³=125 sub voxels is 0.5%, both of which are greater than the error of 0.2% for the randomized sampling method with just 7 random samples. For the cylinder, the randomized sampling method with 7 samples performs nearly as well as the subdivision method with 5³=125 sub voxels. The simulation experiments showed that the DVH errors depended on the random seed used for generating the randomly spaced point cloud. Accordingly, 25 seed values were used, and the worst result among the 25 seed values for the randomized sampling method is shown in the table of FIG. 6B. It was noted that as the size of the randomly spaced point cloud increased, the dependency of the DVH error on the random seed declined. That is, the larger the size k of the point cloud (i.e., the more samples taken per voxel), the less effect the random seed had on the resultant DVH.

### Method of Generating a Bounded DVH

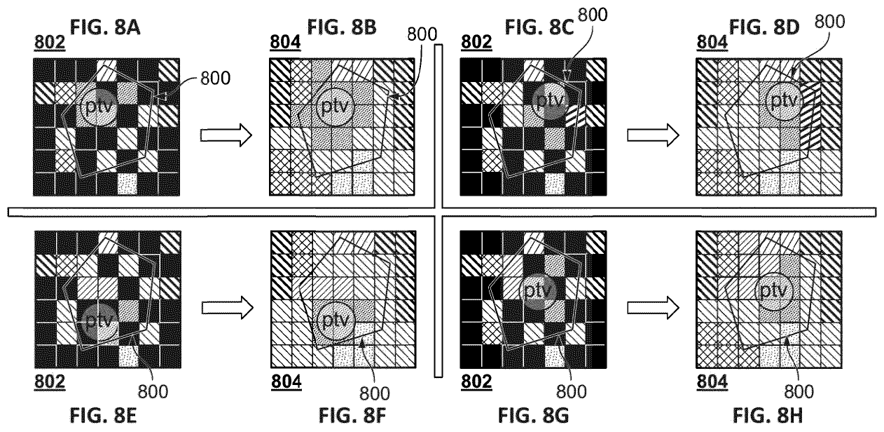

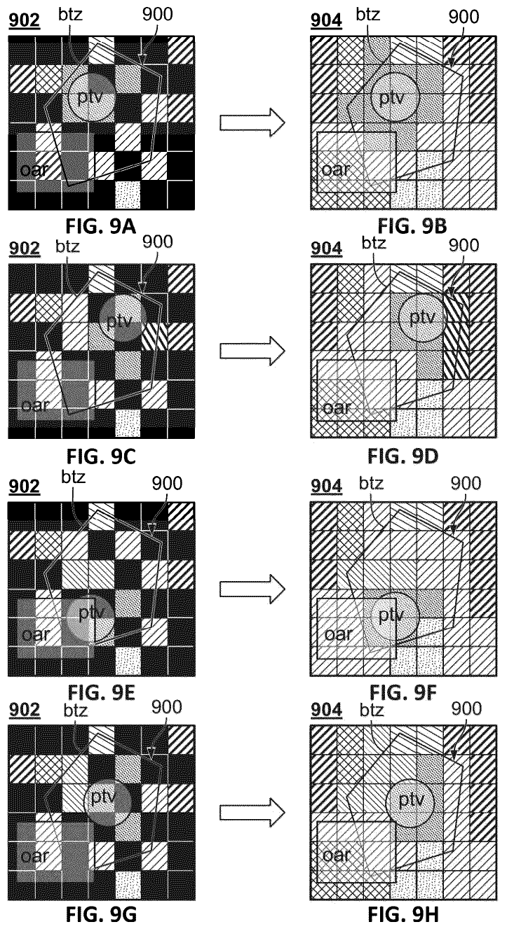

The randomized sampling methods and dual-interpolation methods described herein may be used, alone or in combination, with each other and/or other methods described herein to generate a bounded DVH (bDVH) for one or more VOIs. As described above, a bDVH has an upper bound curve and a lower bound curve that define a range of dose distributions for a VOI (e.g., target region, OARs) and/or the dose variation for the VOI. The upper bound may be a maximum DVH curve, and the lower bound may be a minimum DVH curve. In some variations, a family of DVH curves may be generated by simulating a variety of possible scenarios or changes for the VOI (e.g., a target region and/or OAR within the VOI, and/or the entire VOI), including but not limited to one or more of the following factors: VOI motion (i.e., location changes), VOI size changes, VOI shape changes, VOI orientation changes, and/or PET tracer uptake and distribution variability in the VOI in the case of BgRT. A simulation may include changes to one or more factors about the VOI and/or patient condition, a calculation of the resultant dose distribution to the patient (including the VOI(s)), and a DVH of the VOI. A family of DVH curves for a VOI (and/or one or more target regions and/or OARs of the VOI) and/or may be generated by repeated simulations with different changes to different factors. Because multiple DVH curve calculations are needed for each of these simulations, for one or more VOIs and/or target regions, the randomized sampling methods and/or dual-interpolation methods described herein may help reduce the computational intensity and time to calculate this family of DVH curves for generating the bDVH. In particular, with multiple simulations of different VOI or target region scenarios, it may be time consuming and/or computationally intensive to calculate a full dose image where there are dose values for each voxel. It may be more expedient and computationally feasible to simulate the different scenarios with incomplete dose images, and then use the methods described herein to generate DVHs quickly and with accuracy that is comparable to the accuracy of using a full dose image. FIGS. 8A-8H and 9A-9H depict examples of calculating a family of DVH curves for a target region moving within a VOI. The maximum DVH curve (e.g., right-most curve with higher dose values) in the family of DVH curves may be selected to be the upper bound curve of a bDVH for the target region and the minimum DVH curve (e.g., left-most curve with lower dose values) in the family of DVH curves may be selected to be the lower bound curve of the bDVH. The randomized sampling and dual-interpolation methods described herein may be used in these DVH calculations.

FIGS. 8A, 8C, 8E and 8G depict a dose image 802 with missing or incomplete dose information and the contour of a VOI 800 overlaid on the dose image 802. The black voxels in FIGS. 8A, 8C, 8E and 8G represent voxels with missing or incomplete dose values, while the striped or shaded voxels represent voxels with known dose values. In this example, the VOI includes a PTV (or target region) and in the simulation, the location of the PTV within the VOI contour 800 may change. In other simulations, the size, shape, and/or PET tracer avidity (i.e., tracer uptake, PET imaging data intensity, etc.), alone or in combination, may be changed to generate a set of DVH curves. FIGS. 8A, 8C, 8E and 8G depict the PTV at 4 different locations within the VOI contour 800. A bDVH that captures the range of dose distributions that result from these PTV shifts may be generated by using the dual-interpolation method described above in FIG. 7B to generate a complete dose image 804 (which may also be referred to as an interpolated dose image) using nearest neighbor interpolation and then to assign dose values to points in the VOI point cloud using linear interpolation. FIGS. 8B, 8D, 8F and 8H depict examples of a complete dose image 804 where there are dose values for each voxel of the VOI that have been calculated using nearest neighbor interpolation based on the known dose values in the initial dose image 802 and the location of the PTV. As described above, some variations may use linear interpolation on the complete dose image 804 to generate dose values for the points in the VOI point cloud for any of the randomized sampling methods described herein to generate a DVH curve for each simulated position of the PTV within the VOI. In the example depicted in FIGS. 8A-8H, there are 4 PTV shift locations, so the method may generate 4 DVH curves, and the bDVH curves may comprise the maximum DVH curve of the 4 DVH curves and the minimum DVH curve of the 4 DVH curves. Optionally, the bDVH curves may comprise a nominal DVH curve. In some variations, the nominal DVH curve may be an average or center of the maximum and the minimum DVH curves, and/or may be the DVH curve of the base scenario where the PTV did not shift and/or the DVH curve where the VOI and/or target regions and/or OARs match their planned locations.

FIGS. 9A-9H depict another example of a simulation for generating a bDVH that is similar to the simulation depicted in FIGS. 8A-8H, but includes an OAR in addition to a PTV or target region. In this variation, the simulation generates a bDVH for the PTV and a bDVH for the OAR. The dual-interpolation method and randomized sampling methods described herein may be used to generate DVH curves and bDVH curves for a scenario with a PTV and OAR in a VOI (which may be a biology tracking zone or BTZ in the case of BgRT). FIGS. 9A, 9C, 9E and 9G depict a dose image 902 with missing or incomplete dose information and the contour of a VOI (e.g., biology tracking zone or BTZ 900) overlaid on the dose image 902. The black voxels in FIGS. 9A, 9C, 9E and 9G represent voxels with missing or incomplete dose values, while the striped or shaded voxels represent voxels with known dose values. In this example, the VOI may be a biology tracking zone (BTZ) that includes a PTV (or target region) and an organ at risk (OAR). In the simulation, the location of the PTV within the BTZ contour 900 may change. The location of the OAR relative to the BTZ 900 does not change in this simulation, but in other variations, a simulation may also include OAR location changes. FIGS. 9A, 9C, 9E and 9G depict the PTV at 4 different locations within the BTZ contour 900. A bDVH for the PTV and a bDVH for the OAR that capture the range of dose distributions that result from these PTV shifts may be generated by using the dual-interpolation method described above to generate a complete dose image 904 using nearest neighbor interpolation and then to assign dose values to points of a VOI point cloud using linear interpolation. FIGS. 9B, 9D, 9F and 9H depict examples of a complete dose image 904 (which may also be referred to as an interpolated dose image) where there are dose values for each voxel of the VOI that have been calculated using nearest neighbor interpolation based on the known dose values in the initial dose image 902 and the location of the PTV and optionally, the OAR. As described above, some variations may use linear interpolation on the complete dose image 904 to calculate the dose values for each point of the VOI point cloud for any of the randomized sampling methods described herein to generate a DVH curve for the PTV and a DVH curve for the OAR for each simulated position of the PTV within the BTZ. In the example depicted in FIGS. 9A-9H, there are 4 PTV shift locations, so the method may generate 4 DVH curves for the PTV and 4 DVH curves for the OAR. The bDVH curves for the PTV may comprise the maximum DVH curve of the 4 DVH curves and the minimum DVH curve of the 4 DVH curves for the PTV. The bDVH curves for the OAR may comprise the maximum DVH curve of the 4 DVH curves of the OAR and the minimum DVH curve of the 4 DVH curves for the OAR. Optionally, the bDVH curves for the PTV and the bDVH curves for the OAR may each comprise a nominal DVH curve. In some variations, the nominal DVH curve may be an average or center of the maximum and the minimum DVH curves, and/or may be the DVH curve of the base scenario where the PTV did not shift and/or the DVH curve where the VOI and/or target regions and/or OARs match their planned locations.

While certain variations are described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive variations described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive variations described herein. It is, therefore, to be understood that the foregoing variations are presented by way of example only and that, within the scope of the appended claims and equivalents thereto; inventive variations may be practiced otherwise than as specifically described and claimed. Inventive variations of the present disclosure are directed to each individual feature and/or method described herein. In addition, any combination of two or more such features and/or methods, if such features and/or methods are not mutually inconsistent, is included within the inventive scope of the present disclosure.

The above-described methods can be implemented in any of numerous ways. For example, at least some methods of the present technology may be implemented using hardware, firmware, software, or a combination thereof. When implemented in firmware and/or software, the firmware and/or software code can be executed on any suitable processor or collection of logic components, whether provided in a single device or distributed among multiple devices.

In this respect, various aspects described herein may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other non-transitory medium or tangible computer storage medium) encoded with one or more programs that, when executed on one or more computers or other processors, perform methods that implement the various embodiments of the invention discussed above. The computer readable medium or media can be transportable, such that the program or programs stored thereon can be loaded onto one or more different computers or other processors to implement various aspects of the present invention as discussed above.

The terms “program” or “software” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods disclosed herein need not reside on a single computer or processor but may be distributed in a modular fashion amongst a number of different computers or processors to implement various aspects of the inventions disclosed herein.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in different variations.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

Also, the acts performed as part of the method may be ordered in any suitable way. Accordingly, various methods may be constructed in which acts are performed in an order different than illustrated, which may include performing some acts simultaneously, even though shown as sequential acts in illustrative examples.

1. The method of claim 36, wherein generating the VOI point cloud comprises
generating a randomly spaced point cloud of arbitrary size for each voxel of the VOI and selecting points from the randomly spaced point clouds of each voxel that are within the VOI contour.

2. The method of claim 1, wherein generating the randomly spaced point cloud of arbitrary size for each voxel of the VOI comprises selecting a point cloud size k and assigning the k points of the point cloud to random locations within the voxel.

3. The method of claim 2, wherein assigning the k points of the point cloud to random locations comprises generating random locations from a random seed.

4. The method of claim 3, wherein each voxel has a different random seed, and the random locations of the k points are different for each voxel.

5. The method of claim 2, wherein k is an integer greater than or equal to 1.

6. The method of claim 36, wherein generating the VOI point cloud further comprises using point-in-polygon testing to exclude points outside the VOI contour.

7. The method of claim 6, wherein generating the VOI point cloud further comprises identifying border voxels that are along a boundary of the VOI contour and applying point-in-polygon testing to the border voxels.

8. The method of claim 36, wherein obtaining dose values at each point in the VOI point cloud uses a dose image.

9. The method of claim 36, wherein calculating the dose value for the points in the VOI point cloud comprises interpolating a dose volume in a dose image.

10. The method of claim 8, wherein the dose image comprises a regular grid having dose values at each grid node, and wherein calculating the dose value for the points in the VOI point cloud comprises
generating an interpolated dose image by interpolating dose values for regions between the grid nodes, and
assigning a dose value to each point in the VOI point cloud based on its location on the interpolated dose image.

11. The method of claim 10, wherein interpolating dose values for regions between the grid nodes uses nearest neighbor interpolation.

12. The method of claim 10, wherein the regular grid corresponds with a DICOM CT image grid.

13. The method of claim 10, wherein the regular grid has a coordinate origin.

14. The method of claim 10, wherein voxels of the VOI voxels are registered to the regular grid of the dose image.

15. The method of claim 10, wherein the VOI contour is registered to the regular grid of the dose image.

16. The method of claim 36, wherein generating the DVH curve comprises plotting the proportion of sampled points at the discrete dose levels.

17. The method of claim 36, further comprising generating a graphical user interface that comprises a plot of the DVH curve.

18. The method of claim 17, wherein the graphical user interface comprises the DVH curve overlaid on a bounded DVH, wherein the bounded DVH has a minimum DVH curve and a maximum DVH curve.

19. The method of claim 18, further comprising comparing the DVH curve with the bounded DVH and generating a notification that indicates whether the DVH curve is within a range defined by the minimum DVH curve and the maximum DVH curve.

20-35. (canceled)

36. A method of generating a dose volume histogram (DVH) curve for a volume of interest (VOI) defined by a VOI contour, the method comprising:
generating a VOI point cloud comprising a randomly spaced point cloud of arbitrary size with uniform density for the VOI;
obtaining dose values at each point in the VOI point cloud; and
generating a DVH curve by calculating a proportion of points in the VOI point cloud at discrete dose levels.

37. The method of claim 36, wherein the VOI point cloud comprises a plurality of points within the VOI contour.

38. The method of claim 36, wherein obtaining dose values at each point in the VOI point cloud comprises using a grid of dose values.
